(19) United States
(12) Patent Application Publication
(10) Pub. No.: US 2022/0175658 A1
(43) Pub. Date: Jun. 9, 2022

(54) SKIN CARE OIL

(71) Applicant: OC COSMETICS, Miami, FL (US)

(72) Inventor: Orianne Collins, Miami, FL (US)

(21) Appl. No.: 17/112,764

(22) Filed: Dec. 4, 2020

(51) Int. Cl. A61K 8/9794; A61K 8/9789; A61Q 19/00; A61K 8/19; A61K 8/36; A61K 8/67; A61K 8/65; A61K 8/98; A61K 8/49; A61K 8/92

(52) U.S. Cl. CPC A61K 8/9794; A61K 8/9789; A61Q 19/007; A61K 8/19; A61K 8/36; A61K 8/922; A61K 8/65; A61K 8/988; A61K 8/4953; A61K 8/981; A61K 8/925; A61K 8/678

(57) ABSTRACT

A skin care moisturizing, exfoliating and light reflecting oil includes a plant extract base, for example a composition of coconut oil and hibiscus oil which accounts for less than half of the total volume of the skin care oil. The moisturizing skin care oil also includes one or more essential oils disposed in the plant extract base which essential oils contain essential fatty acids while providing at least one skin softening emollient. Importantly, the moisturizing skin care oil includes an exfoliating and light reflecting powder dispersed within the essential oils. Finally, the moisturizing skin care oil includes a stabilizer, such as vitamin E.

### BACKGROUND OF THE INVENTION

### Field of the Invention

The present invention relates to the field of skin care products and more particularly to a skin care formulation.

### Description of the Related Art

The skin as an organ of the human body, is largest of all organs of the human body. Along with hair, nails, glands and nerves, skin is part of the integumentary system acts as a protective barrier between the outside and the inside of the body. Human skin is composed of three layers of tissue: the epidermis, dermis and hypodermis. As to the outer, epidermal layer, the epidermis is constantly renewed as dead skin cells are shed on a daily basis. The main functions of the epidermal layer include the generation of new skin cells as a replacement for dead, older cells found on the surface of the epidermal layer. Another function of the epidermal layer includes providing the color of the skin in terms according to underlying pigment. Finally, the epidermal layer has the important function of protecting the entirety of the skin organ utilizing keratin, a protein made by cells found in the epidermis, which provide toughness and strength to the skin and also which protect the skin from becoming overly dry.

As it has been long understood, proper skin care is essential to maintaining the health and vitality of this protective organ. To properly care for skin, dermatologists generally recommend the frequent ingestion of water, thorough cleansing, balanced nutrition, toning following cleansing using a formulated toner or astringent to remove fine traces of oil, dirt and make-up that may have been missed while cleansing and moisturizing. Importantly, the addition of an oil layer post-moisturization can be of great importance. In this regard, in small doses, oils are known to make skin appear radiant and have proven effective in mimicking sebum production in the skin—a natural protein charged with preventing too much water from entering the body and preventing too much water from escaping through the epidermal barrier while also protecting the skin from bacterial and fungal infections.

However, oftentimes, consumers avoid the use of oils, particular with respect to the skin of the face, because of the misconception that facial oils compound the problem of dirt retention within the pores of the skin. As well, primarily, most skin care oils produced today are simple presentations of different types of plant oils, known as “essential oils” or synthetic oils such as mineral oil. To wit, most oils are moisturizing oils, of a single type, such as argan, jojoba, rosehip, or fig seed. None are viewed as skin restoration elements like the case with cream-based skin care moisturizers. Therefore, consumers oftentimes forfeit the benefits of a moisturizing oil compound.

### BRIEF SUMMARY OF THE INVENTION

Embodiments of the present invention address deficiencies of the art in respect to skin care the inherent properties of a skin care oil, the embodiments providing a novel and non-obvious skin care moisturizing, exfoliating and light reflecting oil. In an embodiment of the invention, a moisturizing skin care oil includes a plant extract base, for example a composition of coconut oil and hibiscus oil which accounts for less than half of the total volume of the skin care oil. The moisturizing skin care oil also includes one or more essential oils disposed in the plant extract base which essential oils contain essential fatty acids while providing at least one skin softening emollient. Importantly, the moisturizing skin care oil includes an exfoliating and light reflecting powder dispersed within the essential oils. Finally, the moisturizing skin care oil includes a stabilizer, such as vitamin E.

In one aspect of the embodiment, the essential oils include at least one skin softening emollient. For instance, the skin softening emollient may be a vegetable oil such as grape seed oil, avocado oil or jojoba oil. As well, the essential oils may include less than one-third of a total volume of the skin care oil which consisting essentially of almond oil, apricot oil, avocado oil, coconut oil, emu oil, hemp oil, jojoba oil, magnesium oil and grape seed oil in equal volumetric parts. Even further, at least one humectant may be added to the skin care oil, such as a collagen serum or honey extract. Even yet further, an anti-inflammatory may be added to the skin care oil such as an anti-oxidant enriched oil like caffeine oil.

In another aspect of the embodiment, the exfoliating and light reflecting powder is diamond dust. However, in yet another aspect of the embodiment, to limit the exfoliation of the exfoliating and light reflecting while maintaining light reflective properties, the powder may also include gold powder in equal volume as the diamond dust. Even further, so as to institute a preservative within the moisturizing skin care oil, the exfoliating and light reflecting powder additionally includes potassium sorbate powder in equal volume as the diamond dust and the gold powder.

Based upon the composition of the moisturizing skin care oil, several skin care benefits are realized including healing, rejuvenation, firming, sliming, anti-cellulite, toning, restoring, enhancing, shimmering, renewing and anti-aging.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a schematic illustration of a skin care moisturizing, exfoliating and light reflecting oil.

### DETAILED DESCRIPTION OF THE INVENTION

Embodiments of the invention provide for a skin care moisturizing, exfoliating and light reflecting oil. The skin care oil includes a plant extract base which accounts for less than half the total volume of the skin care oil and can include, by way of example, a composition of coconut oil and hibiscus oil. The skin care oil also includes one or more essential oils disposed in the plant extract base. The essential oils contain essential fatty acids while providing at least one skin softening emollient, such as a volumetrically equal combination of jojoba oil and grape seed oil. Importantly, the moisturizing skin care oil includes an exfoliating and light reflecting powder dispersed within the essential oils. The powder provides ample but regulated exfoliation properties, regulated by a non-exfoliating light reflective gold powder, and a preservative for the entirety of the skin care oil, while maintaining the light reflective property expected of a pure diamond dust composition. Finally, the moisturizing skin care oil includes a stabilizer, such as vitamin E.

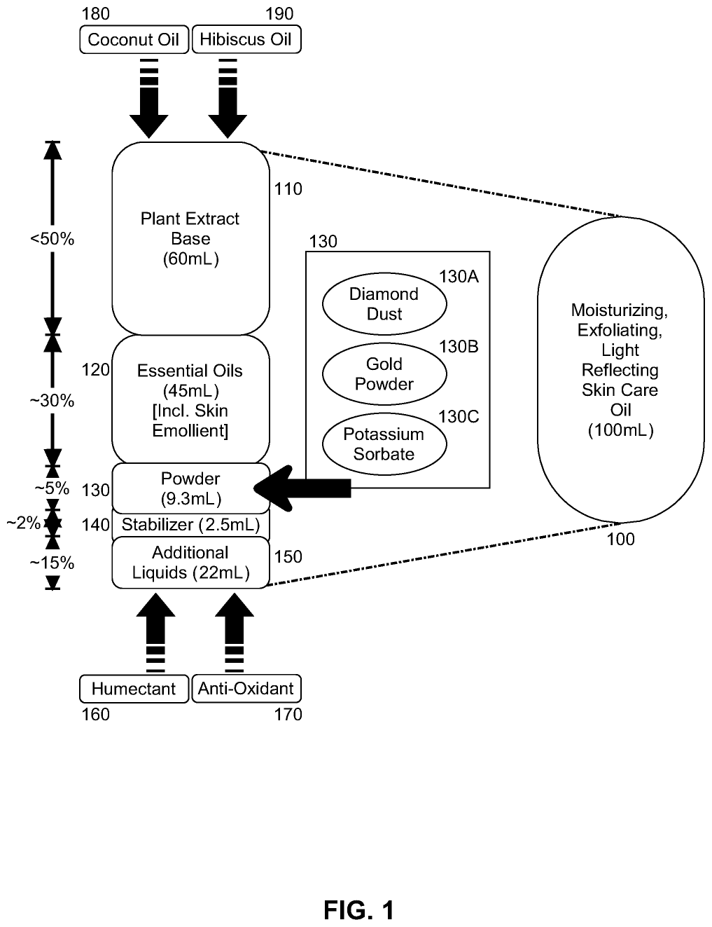

In further illustration, FIG. 1 is a schematic illustration of a skin care moisturizing, exfoliating and light reflecting oil. As shown in FIG. 1 a moisturizing, exfoliating and light reflecting skin care oil 100 includes a plant extract base 110 upon which essential oils 120 are added along with an exfoliating and light reflecting powder 130 and a stabilizer 130, such as Vitamin E. In one aspect of the embodiment, the plant extract base 110 is a combination of coconut oil 180 and hibiscus oil 190 and forms less than half of the total volumetric contribution to the skin care oil 100.

The essential oils 120, which form less than one-third of the total volumetric contribution to the skin care oil 100 include a at least one skin softening emollient such as a vegetable oil. In this regard, in one aspect of the embodiment, the vegetable oil includes a combination of jojoba oil and grape seed oil in equal parts. In another aspect of the embodiment, the essential oils 120 includes the following oils in equal parts: almond oil, apricot oil, avocado oil, coconut oil, emu oil, hemp oil, jojoba oil, magnesium oil and grape seed oil in equal volumetric parts.

Importantly, the exfoliating and light reflecting powder 130 includes diamond dust 130A which acts not only as an exfoliant, but also as a light reflecting agent reflecting light so as to create a “shimmer” on the surface of the skin once applied. However, to avoid over-exfoliation resulting in the damaging of the skin, gold powder 130B is combined with the diamond dust 130A in equal portions so as to enhance the light reflective quality of the skin care oil 100 without increasing the exfoliating qualities of the skin care oil 100 already present owing to the diamond dust 130A. Finally, potassium sorbate 130C is included in equal portions to the diamond dust 130A and the gold powder 130B so as to act as a preservative for the skin care oil 100 thereby increasing the shelf life of the skin care oil 100 and extending the expiration date of the skin care oil 100.

Finally, one or more additional liquids 150 may be added to the skin care oil 100. The additional liquids 150 can include, for instance, a humectant 160. For instance, the humectant 160 can be a collagen serum or a honey extract, either of which are adapted to retain moisture in the epidural layer. As well, the one or more additional liquids 150 can include an anti-inflammatory anti-oxidant enriched oil 170 such as caffeine oil. As it will be apparent to one of skill in the art, while the volumetric total of the plant extract base 110, essential oils 120, exfoliating and light reflecting powder 130, stabilizer 140 and additional liquids 150 exceed the one-hundred milliliters (100 mL) of the skin care oil 100, it is to be appreciated that the exfoliating and light reflecting powder 130 absorb enough of the plant extract base 110, essential oils 120 and additional liquids 150 so as to produce 100 mL of the skin care oil 100.

In even yet further illustration, the following table provides an exemplary composition of the skin care oil 100:

TABLE I
SAMPLE SKIN CARE OIL COMPOSITION

| No. | Ingredients | Measurement | |
|---|---|---|---|
| | BASE: | | |
| 1 | Coconut & | 60 | ML |
| | Hibiscus | | |
| | ESSENTIAL | | |
| | OILS: | | |
| 2 | Almond Oil | 5 | ML |
| 3 | Apricot Oil | 5 | ML |
| 4 | Avocado Oil | 5 | ML |
| 5 | Coconut Oil | 5 | ML |
| 6 | Emu Oil | 5 | ML |
| 7 | Hemp Oil | 5 | ML |
| 8 | Jojoba Oil | 5 | ML |
| 9 | Magnesium | 5 | ML |
| | Oil | | |
| 10 | Grape Seed | 5 | ML |
| | Oil | | |
| | POWDERS: | | |
| 11 | Diamond | 3.1 | ML |
| | Dust | | |
| 12 | Golden Power | 3.1 | ML |
| 13 | Potassium | 3.1 | ML |
| | Sorbate | | |
| | Powder | | |
| | VITAMINS: | | |
| 14 | A | 0.5 | ML |
| 15 | B | 0.5 | ML |
| 16 | C | 0.5 | ML |
| 17 | D | 0.5 | ML |
| 18 | E | 0.5 | ML |
| | ADDITIVES | | |
| 19 | Anti-Cellulite | 0.5 | ML |
| | Oil | | |
| 20 | Anti-Aging | 0.5 | ML |
| | Oil | | |
| 21 | Caffeine Oil | 0.5 | ML |
| 22 | Calming Oil | 0.5 | ML |
| 23 | Chamomile | 0.5 | ML |
| | Oil | | |
| 24 | Cinamon Oil | 0.5 | ML |
| 25 | Collagen | 0.5 | ML |
| | Serum | | |
| 26 | Comfort and | 0.5 | ML |
| | Ease Oil | | |
| 27 | Elastin Oil | 0.5 | ML |
| 28 | Elevation Oil | 0.5 | ML |
| 29 | Energizing | 0.5 | ML |
| | Oil | | |
| 30 | Flexibilty Oil | 0.5 | ML |
| 31 | Ginseng | 0.5 | ML |
| | Extract | | |
| 32 | Grapefruit Oil | 0.5 | ML |
| 33 | Guarana | 0.5 | ML |
| | Etract | | |
| 34 | Healing Oil | 0.5 | ML |
| 35 | Honey Extract | 0.5 | ML |
| 36 | Lavender Oil | 0.5 | ML |
| 37 | Lemongrass | 0.5 | ML |
| | Oil | | |
| 38 | Lime Oil | 0.5 | ML |
| 39 | Mango Oil | 0.5 | ML |
| 40 | Muscle | 0.5 | ML |
| | Relaxer Oil | | |
| 41 | Papaya Oil | 0.5 | ML |
| 42 | Peach Oil | 0.5 | ML |
| 43 | Pineapple Oil | 0.5 | ML |
| 44 | Potasisium | 0.5 | ML |
| | Sorbete | | |
| 45 | Purifying Oil | 0.5 | ML |
| 46 | Retinol Oil | 3.1 | ML |
| 47 | Rose Oil | 0.5 | ML |
| 48 | Royal Jelly | 0.5 | ML |
| | Extract | | |
| 49 | Sassy and | 0.5 | ML |
| | Slimming Oil | | |
| | (Doterra) | | |
| 50 | Sambucus | 0.5 | ML |
| | Nigra Flower | | |
| | Extract | | |
| 51 | Seed Extract | 0.5 | ML |
| 52 | Serenity Oil | 0.5 | ML |
| 53 | Slimming Oil | 0.5 | ML |
| 54 | Strength and | 0.5 | ML |
| | Support Oil | | |
| 55 | Sweet Orange | 0.5 | ML |
| | Oil | | |
| 56 | Thightening | 0.5 | ML |
| | Oil | | |
| 57 | Vanilla Oil | 0.5 | ML |
| 58 | Ylang Ylang | 0.5 | ML |
| | Oil | | |

Of note, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “include”, “includes”, and/or “including,” when used in this specification, specify the presence of stated features, elements, and/or components, but do not preclude the presence or addition of one or more other features, elements, components, and/or groups thereof.

The corresponding structures, materials and equivalents of all means in the claims below are intended to include any structure or material in combination with other claimed elements as specifically claimed. The description of the present invention has been presented for purposes of illustration and description; but the description is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

I claim:

1. A moisturizing skin care oil comprising:
a plant extract base;
a plurality of essential oils disposed in the plant extract base and containing essential fatty acids;
an exfoliating and light reflecting powder dispersed within the essential oils; and,
a stabilizer.

2. The skin care oil of claim 1, wherein the plant extract base is a composition of coconut oil and hibiscus oil.

3. The skin care oil of claim 1, wherein the plant extract base comprises less than fifty percent (50%) of a total volume of the skin care oil.

4. The skin care oil of claim 1, wherein the plurality of essential oils comprises at least one skin softening emollient.

5. The skin care oil of claim 1, wherein the at least one skin softening emollient is a vegetable oil.

6. The skin care oil of claim 1, wherein the vegetable oil comprises a combination of jojoba oil and grape seed oil in equal parts.

7. The skin care oil of claim 1, wherein the plurality of essential oils comprises less than one-third of a total volume of the skin care oil and consists essentially of almond oil, apricot oil, avocado oil, coconut oil, emu oil, hemp oil, jojoba oil, magnesium oil and grape seed oil in equal volumetric parts.

8. The skin care oil of claim 1, wherein the exfoliating and light reflecting powder comprises diamond dust.

9. The skin care oil of claim 8, wherein the exfoliating and light reflecting powder further comprises gold powder in equal volume as the diamond dust.

10. The skin care oil of claim 9, wherein the exfoliating and light reflecting powder yet further comprises potassium sorbate powder in equal volume as the diamond dust and the gold powder.

11. The skin care oil of claim 1, wherein the stabilizer comprises vitamin E.

12. The skin care oil of claim 1, further comprising at least one humectant.

13. The skin care oil of claim 1, wherein the humectant comprises collagen serum.

14. The skin care oil of claim 1, wherein the humectant comprises honey extract.

15. The skin care oil of claim 1, further comprising an anti-inflammatory anti-oxidant enriched oil.

16. The skin care oil of claim 15, wherein the anti-inflammatory anti-oxidant enriched oil is caffeine oil.
